(19) United States
(12) Patent Application Publication
(10) Pub. No.: US 2008/0009720 A1
(43) Pub. Date: Jan. 10, 2008

(54) Catheter connector

(75) Inventors: Richard W. Schefelker, Menomonee Falls, WI (US); Gary J. Secora, Menomonee Falls, WI (US); Sachin Vadodaria, Fox Point, WI (US)

Correspondence Address: GE MEDICAL SYSTEM;C/O FOLEY & LARDNER LLP, 777 EAST WISCONSIN AVENUE, MILWAUKEE, WI, 53202-5306, US

(73) Assignee: General Electric Company

(21) Appl. No.: 11/433,299

(22) Filed: May 12, 2006

(51) Int. Cl. A61B 8/00

(52) U.S. Cl. 600/437

(57) ABSTRACT

A system includes a patient bed configured to support a patient in a patient care facility. The system also includes a medical instrument system configured to obtain medical data of the patient. The system also includes an medical instrument system cable coupled to the medical instrument system at one end. The system may include a catheter configured to be inserted into the patient to cooperate with the medical instrument system to obtain the medical data. The system also includes a catheter cable coupled to the catheter at one end and coupled to the medical instrument system cable at another end. The medical instrument system cable and the catheter cable are connected to each other at a site outside a patient sterile zone including the patient bed.

### FIELD OF THE INVENTION

The present invention relates generally to the field of catheter connectors and specifically to catheter connectors outside of a patient sterile zone.

### BACKGROUND OF THE INVENTION

Invasive catheter connectors generally have a short cable that attaches a connector (coupled to an imaging system) to the catheter. This may cause the imaging system to include a long imaging connector cable that attaches the catheter to the imaging system. By using a short cable on the catheter, the connector of the catheter and the mating connector on the cable of the medical instrument system may reside in the sterile zone or field of the patient. This may cause a number of issues. First, the catheter connector and the mating cable connector should be protected with a sheath that is placed over the both cables and the connectors. This may cause unnecessary use of a sheath and an require an extra step by the staff to place the sheath on the cable before the procedure. Second, the catheter and medical instrument cable connector may be obtrusive during the procedure since it is in the sterile field or zone. Third, by having a short catheter connector, the movement and freedom of the physician who is operating the catheter is restricted.

Thus there is a need for a catheter connector and connection method that does not require an extra preparation step before use. There is also a need for a catheter connector and connection method that is not obtrusive during a medical procedure and is outside the sterile field or zone of a patient. There is also a need for a catheter connector and connection method that does not restrict the movement and freedom of a physician performing a medical procedure.

### SUMMARY OF THE INVENTION

One embodiment of the invention relates to a system. The system includes a patient bed configured to support a patient in a patient care facility. The system also includes a medical instrument system configured to obtain medical data of the patient. The system also includes a medical instrument system cable coupled to the medical instrument system at one end. The system also includes a catheter configured to be inserted into the patient to cooperate with the medical instrument system to obtain the medical data. The system also includes a catheter cable coupled to the catheter at one end and coupled to the medical instrument system cable at another end. The medical instrument system cable and the catheter cable are connected to each other at a site outside a patient sterile zone including the patient bed.

Another embodiment of the invention relates to a method of connecting a catheter cable and an medical instrument system cable outside of a patient sterile zone. The method includes the step of connecting a first cable terminal coupled to the catheter cable and a second cable terminal coupled to the medical instrument system cable to each other defining a connector assembly. The method also includes the step of placing the connector assembly outside the patient sterile zone. The connection of the first and second cable terminals will be outside the patient sterile zone.

Another embodiment of the invention relates to a cable for connecting a medical instrument device to a medical data sensor. The cable includes a first conductor pair coupled at one end to the medical data sensor and coupled to a first connector at another end. The cable also includes a second conductor pair coupled at one end to the medical instrument device and coupled to a second connector at another end. The medical instrument device and medical data sensor are connected when the first and second connectors are coupled together and the first conductor pair are configured so that the first connector is outside a sterile zone defined in a patient care facility when the medical instrument device is in use.

### BRIEF DESCRIPTION OF THE DRAWINGS

FIG. 1 is a perspective view of a system including a medical instrument device, catheter, and catheter connector positioned outside a sterile field or zone according to one exemplary embodiment

FIG. 2 is a side view of a portion of the system of FIG. 1 that illustrates greater detail of the catheter connector according to one exemplary embodiment.

### DETAILED DESCRIPTION OF THE PREFERRED EMBODIMENTS

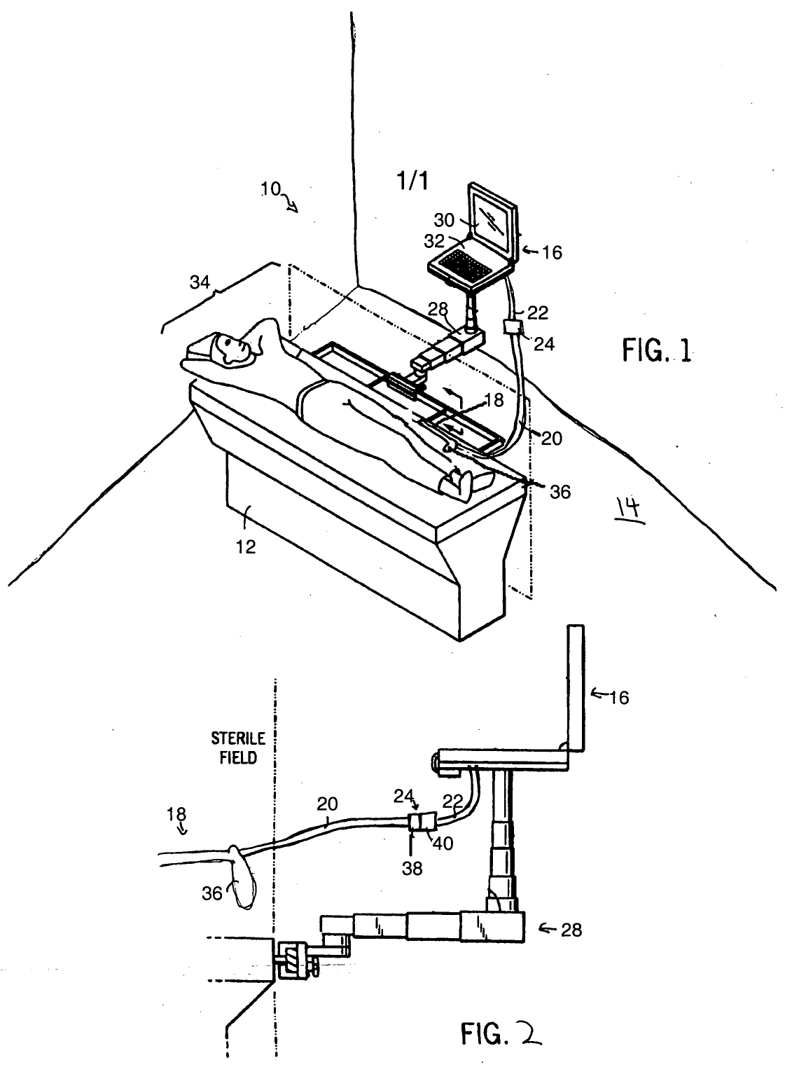

Referring to FIGS. 1 and 2, a system 10 includes a patient bed 12; a patient care facility 14; a medical instrument system or device 16; an imaging sensor 18, for example a catheter; a first conductor pair or catheter cable 20; a second conductor pair or imaging system cable 22, and a connector assembly 24. Patient bed 12 is configured to support a patient 26 in patient care facility 14 so that medical instrument system 16 may obtain medical data of patient 26 using catheter 18 or other suitable sensors. In various exemplary embodiments, patient bed 12 may be any suitable bed of past, present, or future design that is capable of supporting a patient. In one exemplary embodiments, patient 26 may be a human. In other exemplary embodiments, patient 26 may be another anatomical structure such as a dog, cat, horse, or primate. In another exemplary embodiment, patient care facility 14 may be a hospital or hospital room, while in still other exemplary embodiments, facility 14 may be any facility suitable for performing medical procedures, for example imaging, on patient 26.

Medical instrument system or device 16 may include a mounting structure 28 and may cooperate with data sensor or catheter 18 to obtain medical data from patient 26. Medical instrument device 16 may be of a laptop-style configuration with a flip-up screen 30 that displays imaging data or options and a horizontal work area 32. In various exemplary embodiments imaging device 16 may be any imaging device of past, present, or future design such as an ultrasound imaging device, x-ray imaging device, or thermal imaging device. The medical instrument system 16 may also be a hemodynamic or electrophysiology system providing recorded medical data obtained with various medical data sensors. In one example embodiment medical instrument device 16 may be configured to process the data retrieved using an imaging processor.

In one embodiment, mounting structure 28 is configured to support medical instrument device 16 and is coupled to bed 12 via a rail 33 in an area external to a sterile zone or field 34 encompassing bed 12. In another exemplary embodiment, mounting structure 28 may be coupled to bed 12 via means other than a rail. Mounting structure 28 is capable of positioning imaging device 16 in a variety of locations as desired by a clinician for various reasons such as for best viewing or so as to not be obstructing any procedure performed on patient 26. In other exemplary embodiments, mounting structure may be a cart that is not directly coupled to bed 12.

Medical data sensor or catheter 18 may be configured to be inserted into patient 26 to cooperate with medical instrument system 16 and obtain medical data. Catheter 18 may include a handle 36 that allows for gripping and controlling of catheter 18 by a user. In various exemplary embodiments, catheter 18 may be of any past, present, or future design that allows for imaging data retrieval from the interior of patient 26. In another exemplary embodiment, imaging sensor 18 may be configured to take readings externally of patient 26. In another embodiment, the medical data sensor may be configured to obtain hemodynamic or electrophysiology data from the patient 26.

First conductor pair or catheter cable 20 is coupled to catheter 18 at one end and is coupled to medical instrument system cable 22 at another end. Medical instrument system cable 22 and catheter cable 20 are intended to be connected to each other via connector assembly 24 at a site outside of a patient sterile zone or field 34 that includes patient bed 12. In one exemplary embodiment, catheter cable 20 may be longer than medical instrument system cable 22. In another exemplary embodiment, catheter cable 20 may be at least three feet long between the connection between catheter 18 and medical instrument system cable 22. In still another exemplary embodiment, catheter cable 20 may be shorter or the same length as the medical instrument system cable 22 as long as the connection between the two cables is capable of taking place outside of sterile zone or field 34. In various exemplary embodiments, catheter cable 20 may be of any past, present, or future design that facilitates the transfer of medical data, for example imaging data from catheter 18 to medical instrument system 16.

Second conductor pair or medical instrument system cable 22 is coupled at one end to medical instrument system or device 16 and coupled to catheter cable 20 at another end. In various exemplary embodiments, catheter cable 20 may be of any past, present, or future design that facilitates the transfer of medical data from catheter cable 20 to medical instrument system 16.

Connector assembly 24 is configured to couple catheter cable 20 and medical instrument system cable 22 together outside of sterile zone or field 34. A connection outside of sterile zone or field 34 may allow for a health care provider to connect cables 20 and 22 without intruding the sterile field 34. Further, a connection outside of sterile field 34 may allow for an increase of space within sterile field 34. Connector assembly 24 generally includes a first cable terminal or connector 38 and a second cable terminal or connector 40. First connector 38 may be integrally coupled as a single unitary body to catheter cable 20 while second connector 40 may be integrally coupled as a single unitary body to medical instrument system cable 22. Connector assembly may be configured to both attach cables 20 and 22 in order to complete an electrical connection and detach cables 20 and 22 in order to break the electrical connection.

In one exemplary embodiment, connectors 38 and 40 may be embodied as two enclosures, one configured to receive and retain the other in a releasable manner such as with a latch. In this exemplary embodiment, one enclosure may include a number of conducting protrusions that couple with conductors within holes of the other enclosure. In a related exemplary embodiment, the configuration of the two enclosures may be reversed with the opposite enclosure, and thus the opposite cable, receiving the other enclosure and cable. In other exemplary embodiment, connectors 38 and 40 may be of any other connector configuration, such as male and female or plug and socket connectors, that allows for electrical coupling.

Although specific shapes of each element have been set forth in the drawings, each element may be of any other shape that facilitates the function to be performed by that element. For example, connector assembly 24 is shown to have a rectangular prism shape, however, in other embodiments connector assembly 24 may be of another prismatic shape, a spherical shape, or a curvilinear shape.

Although system 10 is illustrated as including multiple features utilized in conjunction with one another, system 10 may alternatively utilize less than all of the noted mechanisms or features. For example, in other exemplary embodiments, bed rail 33 may be omitted with system 10 mounted to bed 12 or other structure via another means.

For purposes of this disclosure, the term “coupled” means the joining of two components (electrical or mechanical) directly or indirectly to one another. Such joining may be stationary in nature or movable in nature. Such joining may be achieved with the two components (electrical or mechanical) and any additional intermediate members being integrally defined as a single unitary body with one another or with the two components or the two components and any additional member being attached to one another. Such joining may be permanent in nature or alternatively may be removable or releasable in nature

The present disclosure has been described with reference to example embodiments, however workers skilled in the art will recognize that changes may be made in form and detail without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example is manifestly intended to be as broad as possible. For example, unless specifically otherwise noted a single particular element may also encompass a plurality of such particular elements.

It is also important to note that the construction and arrangement of the elements of the system as shown in the preferred and other exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes and omissions may be made in the design, operating conditions and arrangement of the preferred and other exemplary embodiments without departing from the spirit of the present subject matter.

What is claimed is:

1. A system comprising:
a patient bed configured to support a patient in a patient care facility;
a medical instrument system configured to obtain medical data of the patient;
a medical instrument system cable coupled to the medical instrument system at one end;
a catheter configured to be inserted into the patient to cooperate with the medical instrument system to obtain the medical data; and
a catheter cable coupled to the catheter at one end and coupled to the medical instrument system cable at another end, wherein the medical instrument system cable and the catheter cable are connected to each other at a site outside a patient sterile zone including the patient bed.

2. The system of claim 1, wherein the catheter cable is longer than the medical instrument cable.

3. The system of claim 2, wherein the catheter cable is at least 3 feet long between the catheter and the medical instrument system cable connection.

4. The system of claim 1, including a connector coupled to one end of each of the catheter cable and medical instrument system cable, with each connector configured to couple with each other.

5. The system of claim 1, wherein the medical instrument is an ultrasound imaging system.

6. A method of connecting a catheter cable and medical instrument system cable outside of a patient sterile zone, the method comprising:
connecting a first cable terminal coupled to the catheter cable and a second cable terminal coupled to the medical instrument system cable to each other defining a connector assembly; and
placing the connector assembly outside the patient sterile zone,
wherein the connection of the first and second cable terminals is outside the patient sterile zone.

7. The method of claim 6, wherein the catheter cable is longer than the medical instrument system cable.

8. The method of claim 7, wherein the catheter cable is at least 3 feet long between the catheter and the first cable terminal.

9. The method of claim 6, including coupling the medical instrument system cable to a medical instrument device.

10. The method of claim 9, wherein the medical instrument device is an ultrasound imaging system.

11. A cable for connecting a medical instrument device to an medical data sensor, the cable comprising:
a first conductor pair coupled at one end to the medical data sensor and coupled to a first connector at another end; and
a second conductor pair coupled at one end to the medical instrument device and coupled to a second connector at another end,
wherein the medical instrument device and medical data sensor are connected when the first and second connectors are coupled together and the first conductor pair are configured so that the first connector is outside a sterile zone defined in a patient care facility when the medical instrument device is in use.

12. The cable of claim 11, wherein the first conductor pair is longer than the second conductor pair.

13. The cable of claim 12, wherein the first conductor pair is at least 3 feet long between the medical data sensor and the first conductor.

14. The cable of claim 11, wherein the medical data sensor is an ultrasound imaging array.
